(19) United States
(12) Patent Application Publication
(10) Pub. No.: US 2016/0331970 A1
(43) Pub. Date: Nov. 17, 2016

(54) Regulation of Protein Levels in Neural Tissue

(71) Applicant: FUNCTIONAL NEUROSCIENCE INC., Toronto (CA)

(72) Inventor: Andres M. Lozano, Toronto (CA)

(73) Assignee: FUNCTIONAL NEUROSCIENCE INC.

(21) Appl. No.: 14/709,086

(22) Filed: May 11, 2015

(51) Int. Cl. A61N 1/36; A61N 1/05

(52) U.S. Cl. CPC A61N 1/36067; A61N 1/0529; A61N 1/36082; A61N 1/36139

(57) ABSTRACT

Techniques are provided for regulating the expression and clearance of proteins in neural tissue using electrical stimulation. The techniques may be used for treating and/or preventing neurodegenerative disorders such as Alzheimer's disease. The treatment involves implanting an electrode within the neural tissue of a human or animal subject, and using the electrode to deliver an electric current to the neural tissue. The voltage, pulse width, frequency, duration, and other parameters of the electrical stimulation may be controlled to provide different effects on protein expression and/or clearance. The position of the electrode may also be selected to control protein expression and/or clearance in a selected neural region.

### FIELD OF THE INVENTION

The present invention relates to techniques for regulating protein levels in neural tissue, and more particularly the use of electrical stimulation to enhance and/or reduce protein levels for the treatment or prevention of neurodegenerative disorders.

### BACKGROUND OF THE INVENTION

Alzheimer's disease is a chronic neurodegenerative disease that accounts for a large percentage of dementia cases. The disease is characterized by the progressive impairment of cognitive functions. The early stages of Alzheimer's disease are often characterized by difficulties with short term memory. As the disease progresses, memory and learning increasingly become impaired, and skills such as speech, reading, writing, planning, and coordinated movement are progressively lost. Behavioural changes such as increased irritability and aggression may also occur. In the final stages, patients are unable to perform even the simplest tasks, and become completely dependent on caregivers.

Neuropathological hallmarks of Alzheimer's disease include the formation of amyloid beta plaques and neurofibrillary tangles. Amyloid beta is a fragment of the amyloid precursor protein, which is a transmembrane protein critical to neuron growth and survival. In Alzheimer's disease, amyloid precursor protein becomes fragmented, and amyloid beta fragments form clumps deposited outside the neurons in dense formations known as plaques. Although the precise role that amyloid beta plaques play in the progression of Alzheimer's disease is not known, it is thought that they may disrupt normal neuron function and ultimately contribute to neuron death.

Neurofibrillary tangles are composed of intracellular hyperphosphorylated tau protein. Tau protein normally stabilises microtubules within neuron cells, which provide a cytoskeleton and transportation system for the cells. In Alzheimer's disease, the tau protein becomes hyperphosphorylated, which causes threads of tau to bind together in tangles, and leads to the destruction of the microtubules. This process is likewise believed to interfere with normal neuron function and contribute to neuron death.

Along with amyloid beta plaques and neurofibrillary tangles, Alzheimer's disease is characterized by the loss of synapses and neurons. Although the amyloid beta plaques and neurofibrillary tangles are believed to contribute to this loss, there is increasing evidence that neuronal dysfunction may begin before the accumulation of plaques and tangles. For example, it has been shown that several transgenic mouse models of Alzheimer's disease present significant deficits in morphological markers of synaptic integrity and impaired behaviour before the onset of amyloid beta plaque formation (Hsia et al. 1999. “Plaque-independent disruption of neural circuits in Alzheimer's disease mouse models.” Proc Natl Acad Sci USA 96 (6):3228-33; Jacobsen et al. 2006. “Early-onset behavioral and synaptic deficits in a mouse model of Alzheimer's disease.” Proc Natl Acad Sci USA 103 (13):5161-6. doi: 10.1073/pnas.0600948103). Furthermore, various studies have shown that Alzheimer's disease is characterized by declines in synaptic proteins such as Growth Associated Protein 43 and synaptophysin, and neurotrophic proteins such as Brain-Derived Neurotrophic Factor and Vascular Endothelial Growth Factor (Masliah et al. 2001. “Altered expression of synaptic proteins occurs early during progression of Alzheimer's disease.” Neurology 56 (1):127-9; Laske et al. 2007. “BDNF serum and CSF concentrations in Alzheimer's disease, normal pressure hydrocephalus and healthy controls.” J Psychiatr Res 41 (5):387-94. doi: 10.1016/j.jpsychires.2006.01.014; Zhang et al. 2008. “CSF multianalyte profile distinguishes Alzheimer and Parkinson diseases.” Am J Clin Pathol 129 (4):526-9. doi: 10.1309/W01Y0B808EMEH12L; Li et al. 2009. “Cerebrospinal fluid concentration of brain-derived neurotrophic factor and cognitive function in non-demented subjects.” PLoS One 4 (5):e5424. doi: 10.1371/journal.pone.0005424; and Yang et al. 2004. “Co-accumulation of vascular endothelial growth factor with beta-amyloid in the brain of patients with Alzheimer's disease.” Neurobiol Aging 25 (3):283-90. doi: 10.1016/SO197-4580(03)00111-8). These declines in protein levels may contribute to the neurodegeneration and loss of cognitive function experienced by Alzheimer's patients.

Many other neurodegenerative diseases are likwise associated with abnormal protein accumulation or loss. For example, Parkinson's disease is characterized by an abnormal accumulation of the protein alpha-synuclein bound to ubiquitin; in Huntington's disease, mutant huntingtin protein aggregates in clumps that interfere with neuron function; and in prion disease misfolded prion proteins accumulate in the brain.

### SUMMARY OF THE INVENTION

The present invention provides techniques for regulating the expression and clearance of proteins and other molecules in neural tissue using electrical stimulation. In preferred embodiments, the techniques are used for treating and/or preventing neurodegenerative disorders such as Alzheimer's disease. The treatment involves implanting an electrode within the neural tissue of a human or animal subject, and using the electrode to deliver an electrical current to the neural tissue. The voltage, pulse width, frequency, duration, and other parameters of the electrical stimulation may be controlled to provide different effects on protein expression and/or clearance. The position of the electrode may also be selected to control protein expression and/or clearance in a selected neural region. In a preferred embodiment, the electrode is implanted in or adjacent to the fornix, and the electrical stimulation regulates the expression and/or clearance of proteins within the fornix and the adjacent neural structures of the hippocampus.

In some embodiments of the invention, the electrical stimulation is used to reduce the concentration of one or more toxic molecule. This could be useful for the treatment and/or prevention of neurodegenerative diseases characterized by the accumulation of toxic proteins, such as Alzheimer's disease, Parkinson's disease, Huntington's disease, and prion disease. The technique may be used, for example, to reduce the concentration of toxic proteins such as hyperphosphorylated tau, amyloid beta, mutant huntingtin protein, or misfolded prior protein. The concentration of these proteins may be reduced by enhancing clearance of the proteins, or by reducing their production. For example, the electical stimulation may be selected to enhance transport of the toxic proteins out of the neural tissue by for example increasing translocation to the vasculature or cerebrospinal fluid or opening the blood brain barrier, or to activate inflammatory processes, activate microglia and astrocytes and enhance phagocytosis, degradation or proteolysis of the toxic proteins within the neural tissue.

In some embodiments of the invention, the electrical stimulation is used to enhance expression of synaptic proteins in the hippocampus of a human or animal subject, such as Growth Associated Protein 43, synaptophysin and a-synuclein. The electrical stimulation may be selected to simultaneously enhance the expression of neurotrophic proteins such as Brain-Derived Neurotrophic Factor or Vascular Endothelial Growth Factor. The enhanced expression of these proteins may stimulate growth of the hippocampus, and improve hippocampus dependent memory in an Alzheimer's patient.

In a preferred embodiment, the electrical stimulation is delivered in pulses at a voltage of 2.5 V, a pulse width of 90 msec, and a frequency of 130 Hz for about 1 hour. In some embodiments, the voltage may be selected between 0.1 V and 10.0 V; the pulse width may be selected between 10 msec and 300 msec; and the frequency may be selected between 1 Hz and 1000 Hz. The stimulation could be applied for any desired length of time, such as one or more seconds; one or more minutes; one or more hours; one or more days; one or more months; or one or more years. In some embodiments, the electrode is designed to be permanently implanted in the subject's neural tissue, for continuous or intermittent stimulation over a long period of time. This could be useful for the chronic treatment of a neurdegenerative disease such as Alzheimer's disease. In some embodiments, protein levels are periodically or continuously monitored, and the parameters of the electrical stimulation are adjusted in light of the detected protein levels. This could be done, for example, using brain images obtained periodically at doctor's appointments. Alternatively, the electrode could have an associated control and monitoring system that automatically detects protein levels, such as in the patient's plasma or cerebrospinal fluid, and adjusts the stimulation based on the protein levels detected.

Accordingly, in one aspect the present invention resides in a method of reducing the concentration of one or more toxic molecules in neural tissue of a human or animal subject, the method comprising: selecting a neural region where the concentration of the one of more toxic molecules is to be reduced; implanting an electrode into the neural tissue of the subject in or adjacent to the selected neural region; configuring the electrode to deliver an electric current selected to reduce the concentration of the one of more toxic molecules; and delivering the electric current to the neural tissue in the selected neural region through the electrode.

In some embodiments, the method is used to treat or prevent a neurodegenerative disorder selected from the group consisting of Alzheimer's disease, Parkinson's disease, amyotrophic lateral sclerosis, Huntington's disease, post injury neurodegeneration, post stroke neurodegeneration, and prion disease.

The one or more toxic molecules may comprise hyperphosphorylated tau, amyloid beta, synuclein, mutant huntingtin protein, various trinucleotide repeat related proteins or misfolded prion protein.

In some embodiments, the electric current is selected to enhance clearance of the one or more toxic molecules and/or to reduce production of the one or more toxic molecules. The electric current may also be selected to enhance transport of the one or more toxic molecules out of the neural tissue; to enhance inflammation; and/or to open the blood brain barrier of the subject, to enhance clearance of the one or more toxic molecules.

The method may also involve monitoring the concentration of the one or more toxic molecules on an ongoing basis using one or more sensors; and adjusting the electric current in response to feedback from the one or more sensors.

In some embodiments, the electric current is selected to activate microglia and astrocytes; to enhance expression of trophic and synaptic molecules; and to promote clearance of the one or more toxic molecules.

In another aspect, the present invention resides in a method of regulating the clearance of one or more proteins in neural tissue of a human or animal subject, the method comprising: selecting a neural region where the clearance of the one of more proteins is to be regulated; implanting an electrode into the neural tissue of the subject in or adjacent to the selected neural region; configuring the electrode to deliver an electric current selected to regulate the clearance of the one of more proteins; and delivering the electric current to the neural tissue in the selected neural region through the electrode.

The electric current may be selected to reduce or enhance the stability of the one or more proteins; or to reduce or enhance proteolysis of the one or more proteins.

In some embodiments, the electrical current is delivered continuously or intermittently for a period of at least 1 hour, or at least 1 year.

The electrode may be permanently implanted into the neural tissue of the subject for chronic treatment of a neurodegenerative disorder.

In some embodiments, the method further comprises: determining a concentration of the one or more proteins in the neural tissue; and adjusting the delivery of the electric current based on the determined concentration. The concentration of the one or more proteins may be determined, for example, by testing a plasma sample, testing a cerebrospinal fluid sample, or preparing a brain image.

In a further aspect, the present invention resides in a method of enhancing the expression of one or more proteins in neural tissue of a human or animal subject, the method comprising: selecting a neural region where the expression of the one of more proteins is to be enhanced; implanting an electrode into the neural tissue of the subject in or adjacent to the selected neural region; configuring the electrode to deliver an electric current selected to enhance the expression of the one of more proteins; and delivering the electric current to the neural tissue in the selected neural region through the electrode; wherein the one or more proteins comprise Growth Associated Protein 43, synaptophysin, and/or α-synuclein.

In some embodiments, the selected neural region is the fornix.

The electric current may be selected to activate the hippocampus and/or to stimulate growth of the hippocampus.

In one embodiment, the electric current is delivered in pulses at a voltage of 2.5 V, a pulse width of 90 msec, and a frequency of 130 Hz for at least 1 hour.

In a preferred embodiment, the method is used to improve hippocampus dependent memory in an Alzheimer's patient.

### BRIEF DESCRIPTION OF THE DRAWINGS

Further aspects and advantages of the invention will appear from the following description taken together with the accompanying drawings, in which:

FIG. 1 is a schematic illustration of an electric stimulator for use with the techniques of the present invention.

FIG. 2 shows an experimental design for an experiment studying the modulation of protein expression in the rat hippocampus following deep brain stimulation of the fornix. Both animal groups, control (CTL) and stimulated (DBS), underwent bilateral implantation of electrodes in the forniceal area. DBS rats received one hour stimulation whereas CTL rats received no stimulation. Animals were sacrificed at different time-points after the initiation of the stimulation: 1 h (n=8/group), 2.5 h (n=8/group), 5 h (n=4/group) and 25 h (n=4/group).

FIG. 3 shows a histological evaluation of the electrode target area. FIG. 3A provides a schematic representation of the electrode tip in the vicinity of the fornix (F). FIG. 3B provides a representative image showing a coronal brain section with the electrode tip in the vicinity of the fornix (3VC: 3rd ventricule; scale bar=400 mm).

FIG. 4 shows the experimental result that fornix DBS increased cFos level in the hippocampus 2.5 h after the initiation of stimulation. FIG. 4A provides representative Western blots; and FIG. 4B provides quantitative analysis of rat hippocampal cFos protein expression in non-stimulated controls (CTL) and stimulated (DBS) animals. Samples were collected at the indicated time-points. GAPDH was used as a loading control. 1 h and 2.5h: n=8 /group; 5 h and 25 h: n=4/group; Student's t-Test **p<0.01 compared to CTL. Data represented as mean±S.E. FIG. 4C shows cFos positive-cells in the rat hippocampus of non-stimulated (CTL) or stimulated (DBS) animals 2.5 h after the initiation of stimulation (n=3/group; HC: Hippocampus, DG: Dentate Gyrus; dashed scale bar=400 mm; solid scale bars=100 mm).

FIG. 5 shows the experimental result that fornix DBS did not change APP, tau and ptau levels in the hippocampus. Representative Western blots and quantitative analysis of rat hippocampal APP (FIGS. 5A and 5B), tau (FIGS. 5C and 5D) and ptau (FIGS. 5E and 5F) protein expression in non-stimulated controls (CTL) and stimulated (DBS) animals are provided. Samples were collected at the indicated time-points. All samples from a single time-point were loaded on the same SDS-PAGE. Actin or tubulin were used as a loading control. 1 h and 2.5 h: n=8 /group; 5 h and 25 h: n=4/group). Data represented as mean±S.E.

FIG. 6 shows the experimental result that fornix DBS increased mature BDNF and VEGF levels in the hippocampus at 2.5 h. Representative Western blots and quantitative analysis of rat hippocampal BDNF (FIGS. 6A and 6B), VEGF (FIGS. 6C and 6D) and GDNF (FIGS. 6E and 6F) protein expression in non-stimulated controls (CTL) and stimulated (DBS) animals are provided. Samples were collected at the indicated time-points. All samples from a single time-point were loaded on the same SDS-PAGE. Actin or GAPDH were used as a loading control. 1 h and 2.5 h: n=8 /group; 5 h and 25 h: n=4/group; Student's t-Test **p<0.01 and ***p<0.001 compared to CTL. Data represented as mean±S.E.

FIG. 7 shows the experimental result that fornix DBS increased GAP-43, synaptophysin and α-synuclein levels in the hippocampus at 2.5 h. Representative Western blots and quantitative analysis of rat hippocampal GAP-43 (FIGS. 7A and 7B), synaptophysin (FIGS. 7C and 7D) and a-synuclein (FIGS. 7E and 7F) protein expression in non-stimulated controls (CTL) and stimulated (DBS) animals are provided. Samples were collected at the indicated time-points. All samples from a single time-point were loaded on the same SDS-PAGE. Actin, GAPDH or tubulin were used as a loading control. 1 h and 2.5h: n=8 /group; 5 h and 25 h: n=4/group; Student's t-Test *p<0.05 and **p<0.01, compared to CTL. Data represented as mean±S.E.

### DETAILED DESCRIPTION OF THE INVENTION

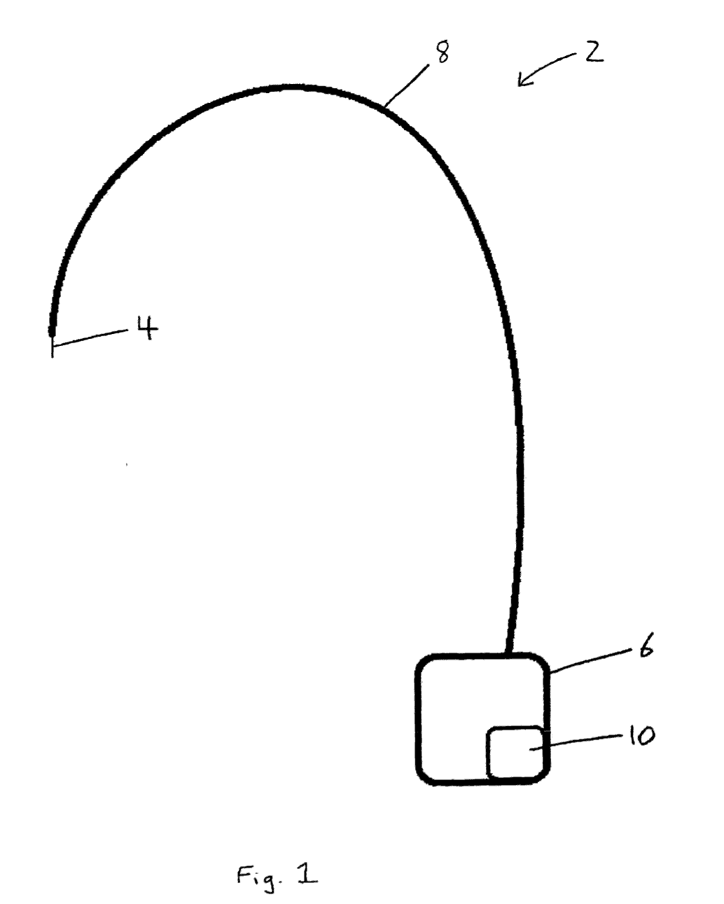

An exemplary electric stimulator 2 for use with the techniques of the present invention is shown in FIG. 1. The stimulator 2 has an electrode 4 that is connected to a pulse generator 6 by an insulated wire 8. The electrode 4 is for implantation within the neural tissue of a patient, and for delivering electric pulses thereto. The electric pulses are generated by the pulse generator 6, and are transmitted through the wire 8 to the electrode 4. The pulse generator 6 has a battery 10, and is programmable to set the parameters of the electric pulses such as voltage, pulse width, frequency, and duration.

The electrode 4 is implanted into the neural tissue of the patient at a location where the concentration of proteins is to be regulated. In a preferred embodiment, the electrode 4 is implanted within or adjacent to the fornix, so that the electric pulses are delivered thereto. The implant procedure may be performed under general anesthesia or with local anesesthia. In some embodiments of the invention, the pulse generator 6 and the wire 8 may also be implanted under the patient's skin. Alternatively, the pulse generator 6 may remain outside of the body, and may for example be held against the body by a strap or in a shirt pocket or the like.

In some embodiments of the invention, the neural region in which the electrode 4 is implanted is selected on the basis of information obtained from a brain scan. For example, a region could be selected where the brain scan reveals the presence of plaque formations, or the loss of neurons and/or synapses.

Once the electrode 4 is implanted, the stimulator 2 is used to deliver electric pulses to the neural tissue. The pulses are selected to affect the concentration of one or more proteins within the neural tissue. For example, the pulses may be selected to enhance the expression of synaptic proteins such as Growth Associated Protein 43 or synaptophysin, for the treatment of a neurodegenerative disease such as Alzheimer's disease. The pulses could also be selected to reduce the concentration of unwanted toxic proteins such as amyloid beta plaques. For example, the pulses could be selected to enhance transport of toxic proteins out of the brain, or to enhance proteolysis of toxic proteins within the brain.

Various parameters of the electric pulses can be adjusted to alter the effect that the pulses have on protein concentrations. For example, a particular combination of voltage, pulse width and frequency may have the effect of enhancing the expression of certain synaptic proteins, while a different combination of voltage, pulse width and frequency may have the effect of enhancing clearance of a toxic protein such as amyloid beta. The pulse generator 6 could be programmed to deliver the particular type of electric pulse that is expected to be most effective at treating the particular condition of the patient. The pulse generator 6 could also be programmed to alternate between different types of pulses, for example to enhance the expression of one type of protein, while enhancing the clearance of another type of protein. The pulse generator 6 may be programmable, for example, via wireless communication with a computer or other control device.

In some embodiments, the concentration of various proteins in the neural tissue is determined, and the pulse parameters are adjusted in light thereof. For example, the patient could periodically undergo brain scans to look for amyloid beta plaques, and the pulses could be adjusted to take into account the degree of plaque formation that is detected. For example, if the plaques are not responding to the treatment, the pulse parameters could be adjusted to try a different combination of voltage, pulse width, frequency and duration. The stimulator 2 could also be associated with a monitoring system that automatically determines the concentration of proteins of interest, and adjusts the pulse parameters accordingly via a feedback loop or a closed loop. For example, the monitoring system could periodically or continuosly monitor the concentration of various proteins in the brain tissue, plasma or cerebrospinal fluid of the patient using an implanted sensor. External sensors such as Positron Emission Tomography (PET) imaging devices or devices that analyze plasma or cerebrospinal fluid samples outside of the body could also be used.

The stimulator 2 could also be programmed to automatically cycle through a variety of different pulse types, and to measure the effect of each pulse type on protein concentrations. A computer learning algorithm could be used to allow the stimulator 2 to recognize and repeat sequences of pulse types that are found to be particularly effective at regulating the concentrations of targeted proteins.

In some embodiments of the invention, the electric current is used to enhance inflammation and/or enhance opening of the blood-brain barrier in the area of the brain where the electrode 4 is implanted. Opening of the blood-brain barrier may be useful for reducing the amount of toxic proteins accumulating in the brain, for example by allowing endogenous and/or exogenous clearing agents to access the toxic proteins. Examples of endogenous clearing agents include immune cells and antibodies, which may be able to target the toxic proteins and selectively remove them. Exogenous clearing agents would include drugs that enhance clearance, for example by binding to the toxic proteins to prevent and/or reverse clumping; to mark the toxic proteins as targets for the immune system; or by enhancing proteolysis. Enhanced inflammation may help to recruit immune cells and related molecules to the area where the toxic proteins are present, and thus enhance the clearance of these proteins by the immune system. The degree of inflammation and/or the movement of molecules and cells through the blood-brain barrier may also be monitored, and the electrical stimulation adjusted accordingly.

In some embodiments of the invention, the electric current is used to enhance stability of selected proteins. For example, the parameters of the electric current may be selected to enhance the expression of heat shock proteins or other chaperone proteins, for the purpose of stabilizing proteins that are at risk of becoming misfolded, such as the prion protein. Heat shock proteins may also help to refold proteins that have become misfolded.

The electric current may also be used to reduce the stability of unwanted proteins. For example, the electric current may be selected to damage or otherwise alter the structure and/or shape of a toxic protein, so that it becomes targeted for proteolysis. The electric current may also be selected to upregulate the expression of proteins such as ubiquitin, for the purpose of marking the toxic proteins for degradation.

Reference will now be made to the following examples, which are provided to give the reader a more complete understanding of the invention, and are not intended to limit the scope of the invention. It is to be appreciated that electrode positions and stimulation parameters other than those described in the examples, including electrode positions and stimulation parameters selected to produce different effects than those described, fall within the scope of the invention.

### Examples

The fornix, a white matter tract bundle, is the predominant efferent projection from the hippocampus to the septal regions and mammillary bodies. Another major component of the fornix is the axonal projections from the septal area to the hippocampus. The fornix constitutes an integral part of the classical circuit of Papez, a major pathway of the limbic system, primarily involved in memory function. Lesions of the fornix in experimental animals and humans are known to produce memory deficits (Tsivilis, D., et al., A disproportionate role for the fornix and mammillary bodies in recall versus recognition memory. Nat Neurosci, 2008. 11(7): p. 834-42; Wilson, C. R., et al., Addition of fornix transection to frontal-temporal disconnection increases the impairment in object-in-place memory in macaque monkeys. Eur J Neurosci, 2008. 27(7): p. 1814-22; and Thomas, A. G., P. Koumellis, and R. A. Dineen, The fornix in health and disease: an imaging review. Radiographics, 2011. 31(4): p. 1107-21).

Deep brain stimulation (DBS) refers to the therapeutic delivery of electrical current through implanted electrodes in precisely targeted areas of the brain. The experiments described herein relate to the use of DBS for treatment of neurodegenerative disorders, and to fornix DBS specifically for treating Alzheimer's disease (AD). In the present study, we investigated the effects of fornix DBS on the modulation of protein expression in the rat hippocampus at different time-points following high frequency fornix stimulation for one hour. We analyzed the expression of selected proteins within 3 broad categories: 1) proteins known to be involved in Alzheimer's disease including tau, phosphorylated tau (ptau), amyloid precursor protein (APP) as well as 2) the trophic factors brain-derived neurotrophic factor (BDNF), glial cell-line derived neurotrophic factor (GDNF) and vascular endothelial growth factor (VEGF) and 3) synaptic markers of long-term potentiation and plasticity, namely synaptophysin and growth associated protein 43 (GAP-43). We also studied the effect of fornix stimulation on the expression of cFos and selected heat shock proteins as markers of neurophysiologic activity and stress.

### Materials and Methods

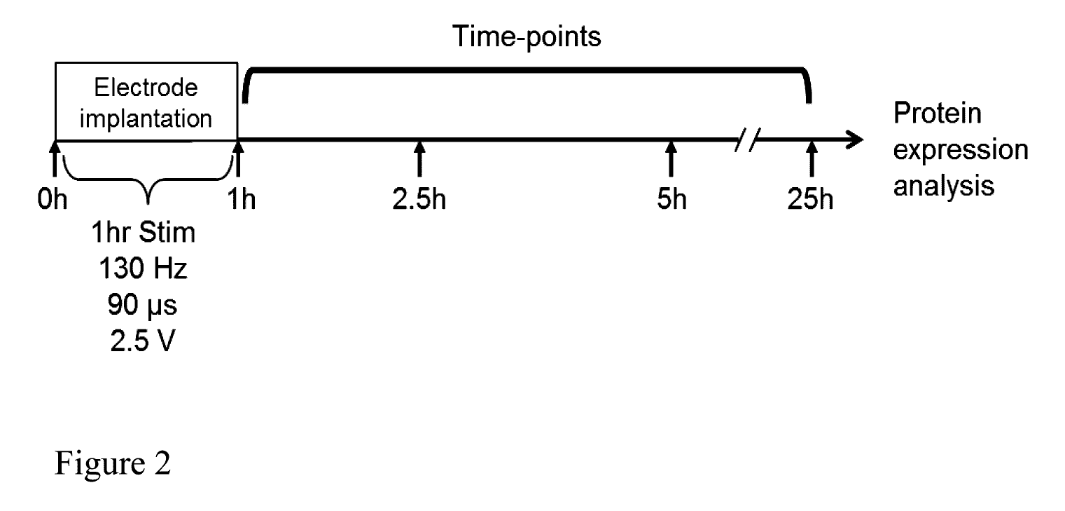

A summary of our experimental design and timeline is outlined in FIG. 2.

### Animals

This study was approved by the Toronto Western Research Institute Animal Care Committee and is in accordance with the guidelines of the Canadian Council on Animal Care. Adult male Wistar rats (270-300 g) were housed with ad libitum access to food and water in a room maintained at a constant temperature (20-22° C.) and on a 12 hour: 12 hour light-dark cycle.

### Electrical Stimulation of the Fornix

Animals were anesthetized with isoflurane and had their heads fixed in a stereotactic instrument (Model 900, David Kopf Instruments). The pre-selected target was the region in close vicinity to the fornix, to avoid damage to the white matter fibers. Platinum concentric bipolar electrodes (model SNEX-100, cathode tip with 100 μm diameter and 0.25 mm of exposed length; Rhodes Medical Instruments) were bilaterally implanted at the following coordinates relative to bregma: anteroposterior −1.8 mm, mediallateral 1.4 mm, dorsoventral 8.2 mm (Paxinos, G. and C. Watson, The Rat Brain in Stereotaxic Coordinates. 2005, Elsevier Academic Press. p. 166). Stimulation was applied with a handheld stimulator (Medtronic 3628 screener) for one hour at parameters that were similar to those in our previous report (2.5 V, 90 μsec of pulse width, 130 Hz frequency) (Hamani, C., et al., Memory rescue and enhanced neurogenesis following electrical stimulation of the anterior thalamus in rats treated with corticosterone. Exp Neurol, 2011. 232(1): p. 100-4; Toda, H., et al., The regulation of adult rodent hippocampal neurogenesis by deep brain stimulation. J Neurosurg, 2008. 108(1): p. 132-8). Control animals had electrodes implanted but did not receive stimulation. Following stimulation, electrodes were removed, the surgical incision was closed and the animals were allowed to recover.

### Tissue Collection

After the initiation of stimulation, animals were euthanized at various time-points: 1 h, 2.5 h, 5 h and 25 h (FIG. 2). Under deep anesthesia, rats were decapitated, brains were quickly removed from the skull and divided in the sagittal plane. Hippocampi were dissected from anterior to posterior (including both dorsal and ventral regions), collected, immediately frozen in dry ice and stored at −80° C. until processed for western blotting.

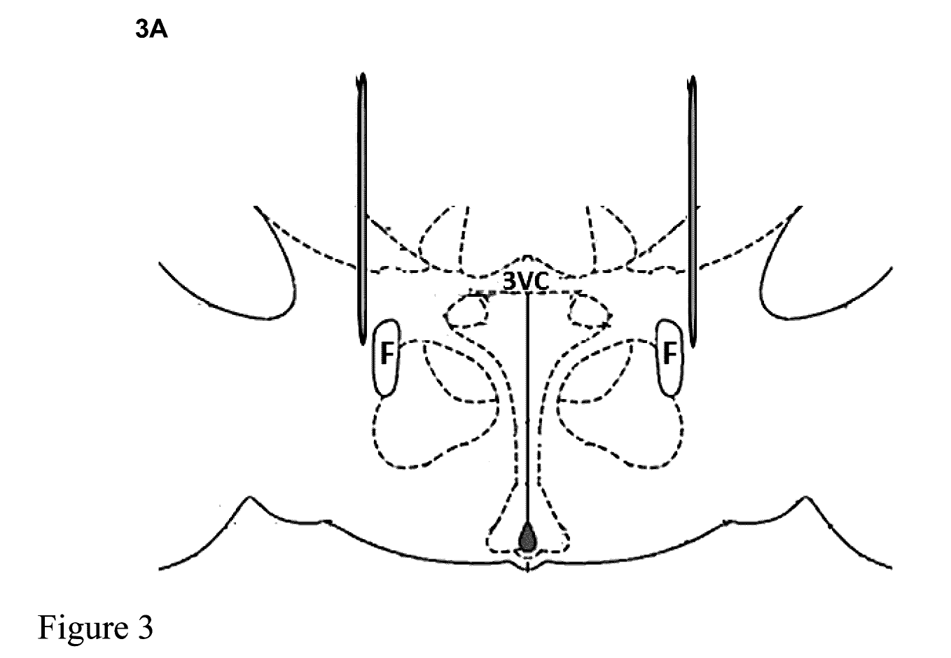

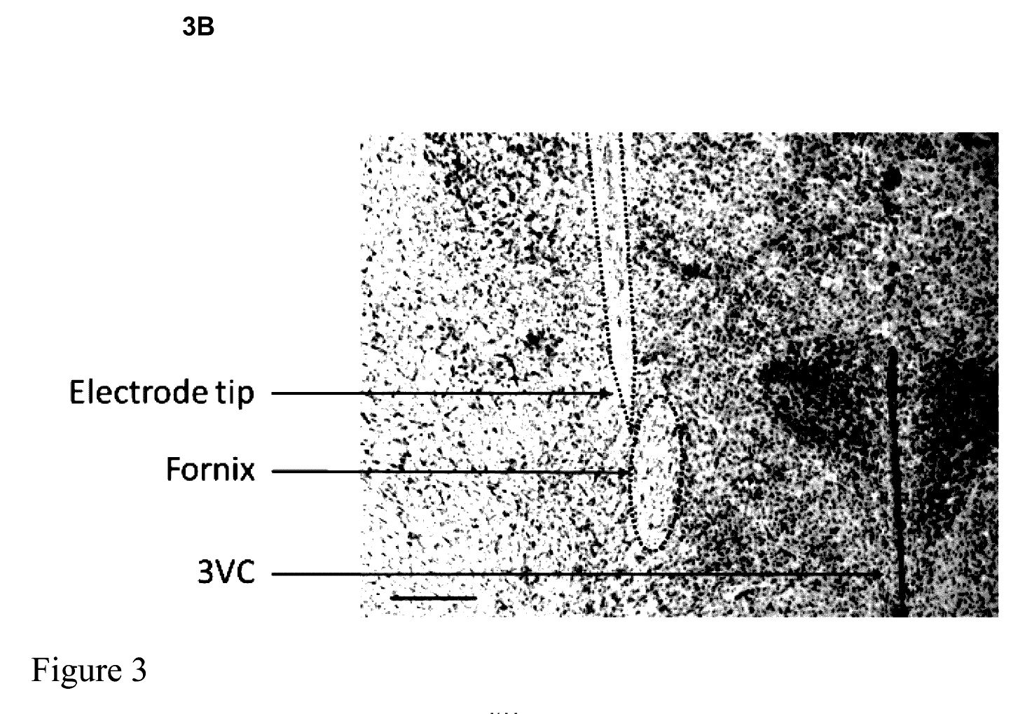

To locate the electrodes' sites, coronal 25 μm sections anterior to the hippocampus were cut on a cryostat and processed for cresyl violet. Only samples from animals with electrodes located within the boundaries of the fornix (<400 μm) were included in the analysis (FIG. 3). A total of 66 rats underwent the surgical procedure: 12 were excluded due to misplaced electrodes. Eight stimulated (DBS) and 8 non-stimulated control (CTL) rats were studied in the 1 h and 2.5 h time-point groups. Four DBS and four CTL animals were studied in the 5 h and 25 h time-point groups. Finally, 3 DBS and 3 CTL animals were perfused 2.5 h after the insertion of the electrodes and studied for cFos staining.

### Western Blot Analysis

### Western Blotting

Samples were homogenized in RIPA lysis buffer (50 mM Tris-HCl, pH 7.4, 2 mM EDTA, pH 8, 150 mM NaCl, 1% Triton X-100, protease inhibitor cocktail [Roche]) on ice for 30 min and then centrifuged (13,000 X g, 15 min, at 4° C.). Protein concentration was determined using the DC protein assay from Biorad. Samples with equal amounts of total protein (30 to 100 μg) were then separated by SDS-PAGE and transferred to PVDF membranes (Roche). After blocking for 30 min in a solution of 0.1 M Trisbuffered saline with 0.1% Tween-20 (TBST) supplemented with 5% non-fat milk for 30 min, membranes were incubated at 4° C. overnight with primary antibodies (see list below). On the following day, membranes were washed three times with TBST, and then incubated with secondary antibodies for 1 h at room temperature. Membranes were then washed three times for 10 min and protein expression was visualized using enhanced chemiluminescence kits (ECL: GE Healthcare or ECL Plus: Thermo Fisher Scientific) followed by exposure to x-ray film for detection. Equal loading of total protein was confirmed using anti-actin, anti-GAPDH, or anti-tubulin antibodies.

### Antibodies

Rabbit monoclonal cFos, synaptophysin and GAPDH antibodies (1:1000; Cell Signaling); Rabbit polyclonal APP, GAP-43, heat shock protein 70 (HSP70) and α/β-tubulin antibodies (1:1000; Cell Signaling); Mouse monoclonal tau and ptau antibodies (1:1000; Cell Signaling); Rabbit polyclonal BDNF and GDNF antibodies (1:1000; Alomone); Rabbit polyclonal VEGF antibody (1:1000; Abcam); Mouse monoclonal α-synuclein antibody (1:1000; BD Biosciences); Rabbit polyclonal HSP40 antibody (1:1000; Stressgen); Mouse monoclonal C-terminus of HSC70-Interacting Protein (CHIP) antibody (1:200; Santa Cruz Biotechnology Inc.); Rabbit actin antibody (1:1000; Sigma Aldrich); Horseradish-peroxidaseconjugated anti-mouse IgG and horseradish-peroxidase-conjugated anti-rabbit IgG (1:5000; GE Healthcare).

### Histology and c-Fos Histochemistry

Six rats (3 DBS and 3 CTL) were studied for cFos histochemistry: 2.5 h after the insertion of the electrodes, animals were deeply anesthetized and transcardially perfused with normal saline, followed by a 4% paraformaldehyde (PFA) solution. Brains were then removed from the skull, fixed overnight in PFA, transferred into 30% sucrose for 3 days at 4° C. and stored at −80° C. Coronal 40 μm sections were cut on a cryostat, pretreated with 0.25% Triton X-100 for 30 min followed by 5% normal goat serum for 30 min. Sections were then incubated with primary rabbit anti-cFos antibody (1:800; Cell Signaling) overnight at 4° C. After 2 h incubation with a secondary antibody (goat biotin-SP anti-rabbit IgG 1:200; Jackson Immuno Research) at room temperature, sections were treated with avidin-biotin complex (Vectastain Elite ABC kit, Vector Labs) for 1 h and visualized with a diaminobenzidine reaction (Vector Labs).

### Data Analysis and Statistics

Western blot bands were quantified using ImageJ software (National Institutes of Health) by analyzing pixel density using rectangular areas of uniform size for each band analyzed. A semiquantitative analysis was performed by densitometry, normalizing protein levels with actin, GAPDH or tubulin. Each band representing the protein of interest was first normalized to the band representing the loading control (actin, GAPDH or tubulin) in the same lane: results were calculated and graphically shown as the ratio of BDNF, GDNF, α-synuclein, CHIP, tau, ptau or HSP70 relative to actin; cFos, VEGF or GAP-43 relative to GAPDH; APP, synaptophysin or HSP40 relative to tubulin. To account for possible differences in intensity levels between the scanned membranes, values were then normalized and expressed as a ratio of the average level of protein in the CTL group for each animal. Experimental data followed a normal distribution, as assessed by the Shapiro-Wilk normality test. Western blot data was analyzed with a Student's t-test with statistical significance set at p<0.05. Results are shown as means±standard error of the mean (SEM).

### Results

We investigated both AD-related and candidate proteins whose levels of expression after DBS could be predicted to change.

### Neuronal Activation Marker: cFos

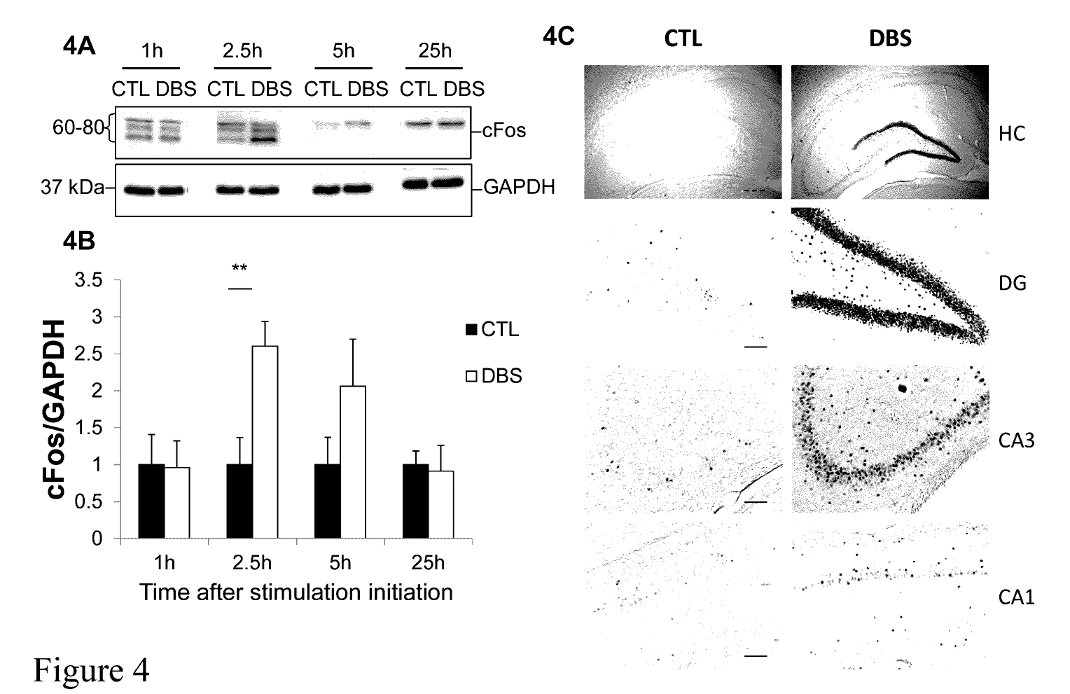

Although cFos expression in the hippocampus was not different between CTL and DBS groups immediately after the fornix stimulation (1 h), there was an increase in cFos at 2.5 h after stimulation was initiated compared to the CTL group (FIG. 4; normalized intensities CTL: 1±0.37 vs DBS: 2.6±0.33, p<0.001). This robust increase is also illustrated by cFos histochemistry staining: cFos expression was strongly elevated in the dentate gyrus granule cell layer at 2.5 h compared to CTL group (FIG. 4C) as well as in CA3 and CA1 layers. By 5 hours and 25 hours after stimulation, cFos levels returned to baseline.

### Selected Proteins Involved in the Molecular Pathogenesis of AD: APP-tau-ptau

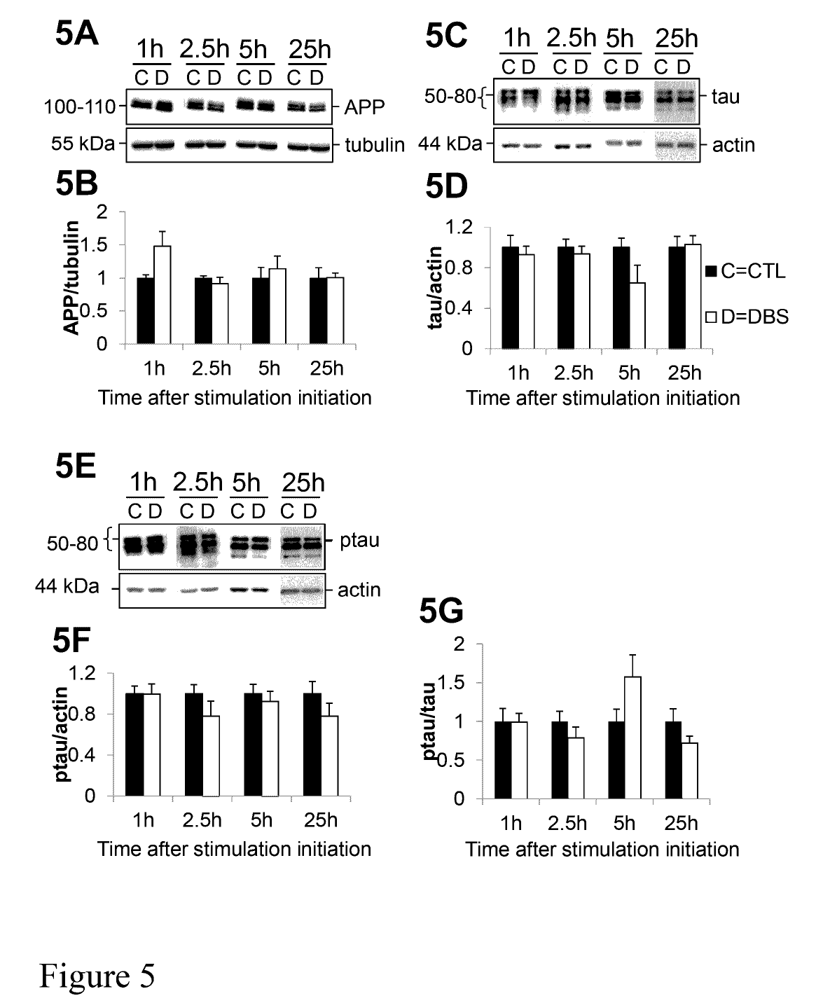

High frequency stimulation of the fornix had no significant effect on the amount of APP, tau and ptau protein expression (FIG. 5). Although APP tended to increase just after the 1 h stimulation, this was not significant compared to CTL (CTL: 1±0.05 vs DBS: 1.48±0.22, p=0.053). No β-Amyloid (Aβ) could be detected in our groups, primarily because young rats have very low concentrations of both Aβ-40 and Aβ-42, and as such were undetectable by western blot (Silverberg, G. D., et al., Amyloid deposition and influx transporter expression at the blood-brain barrier increase in normal aging. J Neuropathol Exp Neurol, 2010. 69(1): p. 98-108; Silverberg, G. D., et al., Amyloid efflux transporter expression at the blood-brain barrier declines in normal aging. J Neuropathol Exp Neurol, 2010. 69(10): p. 1034-43).

### Selected Neurotrophic Factors: BDNF-VEGF-GDNF

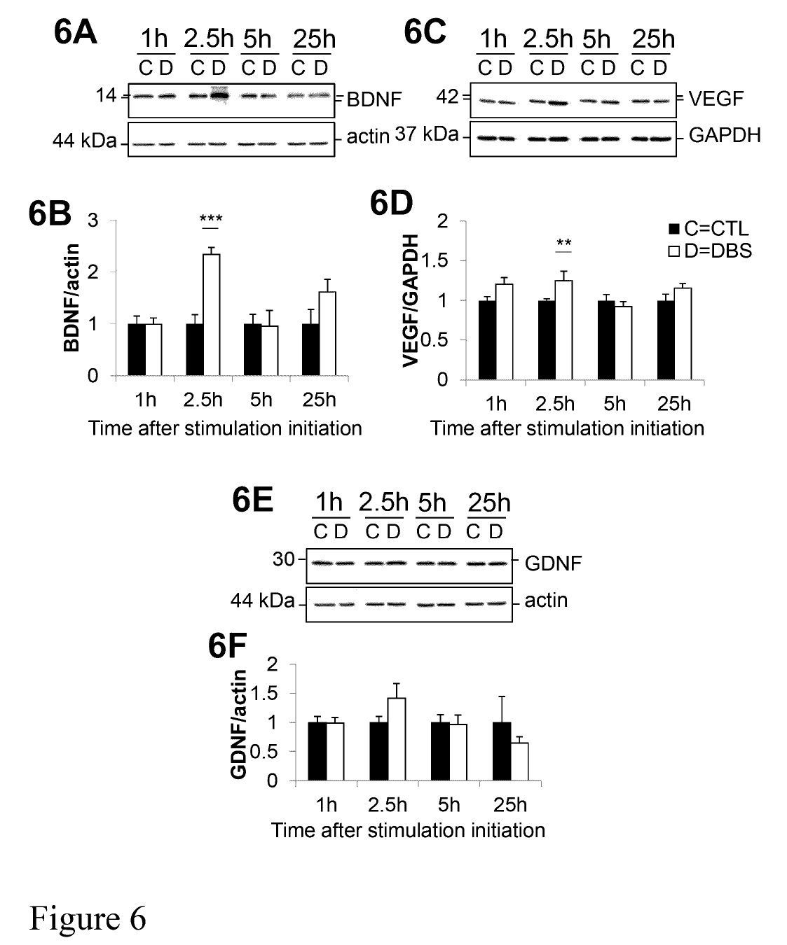

BDNF levels increase of 2.3 fold in the hippocampus at 2.5 h (FIG. 6B; CTL: 1±0.18 vs DBS: 2.34±0.14, p<0.001) when compared to CTL. BDNF expression returned to CTL level at 5 h and remained so at 25 h after stimulation was initiated. VEGF significantly increased at 2.5 h compared to CTL (FIG. 6D; CTL: 1±0.02 vs DBS: 1.25±0.07, p<0.01). Although there was no significant difference, VEGF did show a trend to increase in the hippocampus immediately after the end of the fornix DBS (CTL: 1±0.05 vs DBS: 1.2±0.08, p=0.057). No significant differences were found between CTL and DBS groups at 5 h and 25 h time-points. No significant difference in GDNF expression was observed between CTL and DBS groups. Although GDNF levels tended to be higher in DBS compared to CTL at 2.5 h, this difference was not significant (FIG. 6E; CTL: 1±0.11 vs DBS: 1.42±0.25,p=0.15).

### Synaptic Proteins: GAP-43-synaptophysin-α-synuclein

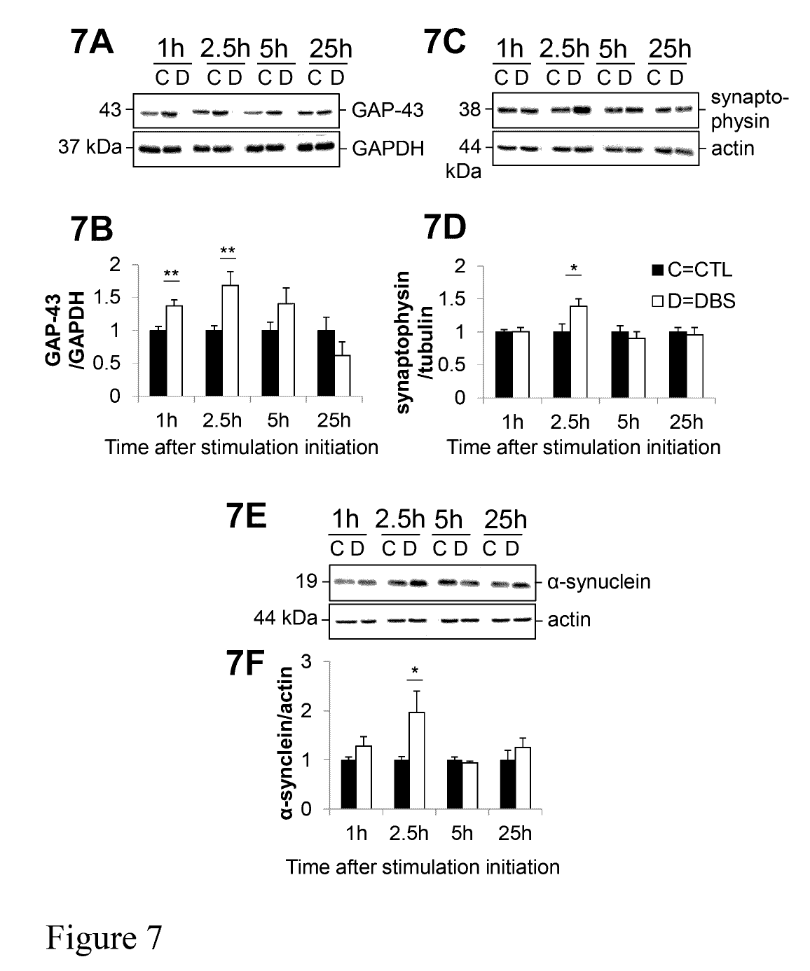

As shown in FIG. 7, rats treated with fornix stimulation had a robust increase in GAP-43 in the hippocampus immediately (1 h) and at 2.5 h (FIG. 7B; p<0.01). Synaptophysin expression was not different between groups immediately after the 1 h stimulation but showed an increase at 2.5 h (FIG. 7D; CTL: 1±0.12 vs DBS: 1.39±0.11, p<0.05). Both GAP-43 and synaptophysin returned to CTL levels at 5 h and 25 h. No differences in hippocampal α-synuclein levels were found immediately after the 1 h stimulation of the fornix. However, DBS rats showed a significant elevation of a-synuclein at 2.5 h compared to the CTL group (FIG. 7F; CTL: 1±0.07 vs DBS 1.97±0.43, p<0.05). Levels returned to baseline at 5 h and 25 h after stimulation.

### Chaperone Proteins

No difference were found in HSP40, HSP70 and CHIP in the hippocampus between CTL and DBS groups following fornix DBS at all studied time-points (data not shown).

### Discussion

Our study shows that one hour of DBS of the fornix area modulates protein expression in the hippocampus, a connected remote area. Acute DBS in the forniceal area increases trophic factors including BDNF and VEGF (FIG. 6) and the synaptic markers GAP-43, synaptophysin and α-synuclein (FIG. 7). Notably, these increases occurred within 2.5 h after the initiation of the fornix stimulation and returned to control levels by 5 h. No changes were found in APP, tau, ptau (FIG. 5), GDNF and chaperone proteins.

We first measured the expression of the activity-regulated gene cFos, a marker for acute neuronal and synaptic activity previously used to study the effects of DBS at cellular levels (Stone, S. S., et al., Functional convergence of developmentally and adult-generated granule cells in dentate gyrus circuits supporting hippocampus-dependent memory. Hippocampus, 2011. 21(12): p. 1348-62; Stone, S. S., et al., Stimulation of entorhinal cortex promotes adult neurogenesis and facilitates spatial memory. J Neurosci, 2011. 31(38): p. 13469-84; Schulte, T., et al., Induction of immediate early gene expression by high-frequency stimulation of the subthalamic nucleus in rats. Neuroscience, 2006. 138(4): p. 1377-85; Saryyeva, A., et al., c-Fos expression after deep brain stimulation of the pedunculopontine tegmental nucleus in the rat 6-hydroxydopamine Parkinson model. J Chem Neuroanat, 2011. 42(3): p. 210-7). Using parameters analogous to clinical high-frequency DBS, we found that at 2.5 h, cFos expression was strikingly elevated and mainly located in the dentate gyrus granular cell layer (FIG. 4) but also in CA1 and CA3 regions, suggesting that stimulation of the fornix led to anterograde transynaptic activation and possibly retrograde backfiring of the hippocampus.

Interestingly, we found that one hour of fornix DBS led to a significant elevation in the hippocampus of two synaptic markers, GAP-43 and synaptophysin (FIG. 7). These molecules are known to play a key role in axonal growth and guidance in addition to synaptic plasticity and synaptogenesis, and are important for memory processing (Aigner, L., et al., Overexpression of the neural growth-associated protein GAP-43 induces nerve sprouting in the adult nervous system of transgenic mice. Cell, 1995. 83(2): p. 269-78; Strittmatter, S. M., et al., Neuronal pathfinding is abnormal in mice lacking the neuronal growth cone protein GAP-43. Cell, 1995. 80(3): p. 445-52; Biewenga, J. E., L. H. Schrama, and W. H. Gispen, Presynaptic phosphoprotein B-50/GAP-43 in neuronal and synaptic plasticity. Acta Biochim Pol, 1996. 43(2): p. 327-38; Rekart, J. L., K. Meiri, and A. Routtenberg, Hippocampal-dependent memory is impaired in heterozygous GAP-43 knockout mice. Hippocampus, 2005. 15(1): p. 1-7; Grasselli, G., et al., Impaired sprouting and axonal atrophy in cerebellar climbing fibres following in vivo silencing of the growth-associated protein GAP-43. PLoS One, 2011. 6(6): p. e2079134-38). AD is characterized by loss of synapses (DeKosky, S. T., S. W. Scheff, and S. D. Styren, Structural correlates of cognition in dementia: quantification and assessment of synapse change. Neurodegeneration, 1996. 5(4): p. 417-21; Hashimoto, M. and E. Masliah, Cycles of aberrant synaptic sprouting and neurodegeneration in Alzheimer's and dementia with Lewy bodies. Neurochem Res, 2003. 28(11): p. 1743-56) and reductions in synaptophysin expression in frontal, parietal, occipital and temporal cortex and hippocampus of patients (Kirvell, S. L., M. Esiri, and P. T. Francis, Down-regulation of vesicular glutamate transporters precedes cell loss and pathology in Alzheimer's disease. J Neurochem, 2006. 98(3): p. 939-50; Head, E., et al., Synaptic proteins, neuropathology and cognitive status in the oldest-old. Neurobiol Aging, 2009. 30(7): p. 1125-34). A post-mortem study found that, compared to control subjects, mild AD cases had a loss of 25% synaptophysin immunoreactivity in the frontal cortex with no change in GAP-43. In advanced disease there was a progressive decline in both synaptic proteins (Masliah, E., et al., Altered expression of synaptic proteins occurs early during progression of Alzheimer's disease. Neurology, 2001. 56(1): p. 127-9). This leads us to speculate that increasing the expression of GAP-43 and synaptophysin as we have seen with DBS, could be beneficial in AD.

Our results also demonstrated that one hour of fornix DBS led to the elevation in neurotrophic factors such as BDNF and VEGF in the hippocampus, 2.5 h after the initiation of fornix stimulation. BDNF plays an important role in neuronal differentiation, neuron survival, synapse formation and regulation of activity-dependent changes in synapse structure and function (Acheson, A., et al., A BDNF autocrine loop in adult sensory neurons prevents cell death. Nature, 1995. 374(6521): p. 450-3; Park, H. and M. M. Poo, Neurotrophin regulation of neural circuit development and function. Nat Rev Neurosci, 2013. 14(1): p. 7-23). BDNF is also a regulator of Long-Term Potentiation (LTP) in the hippocampus (Bramham, C. R. and E. Messaoudi, BDNF function in adult synaptic plasticity: the synaptic consolidation hypothesis. Prog Neurobiol, 2005. 76(2): p. 99-125; Minichiello, L., TrkB signalling pathways in LTP and learning. Nat Rev Neurosci, 2009. 10(12): p. 850-60) and plays a crucial role in learning and memory. Further, recognition memory is associated with increased release of BDNF in the dentate gyrus and the perirhinal cortex (Callaghan, C. K. and Á. Kelly, Differential BDNF signaling in dentate gyrus and perirhinal cortex during consolidation of recognition memory in the rat. Hippocampus, 2012. 22(11): p. 2127-35.48) whereas, hippocampal-specific deletion of the BDNF expression impairs object recognition and spatial learning in the water maze (Heldt, S. A., et al., Hippocampus-specific deletion of BDNF in adult mice impairs spatial memory and extinction of aversive memories. Mol Psychiatry, 2007. 12(7): p. 656-70; Furini, C. R., et al., Beta-adrenergic receptors link NO/sGC/PKG signaling to BDNF expression during the consolidation of object recognition long-term memory. Hippocampus, 2010. 20(5): p. 672-83). Recent studies have reported reduced BDNF in the cerebrospinal fluid (CSF) of AD patients compared to controls (Laske, C., et al., BDNF serum and CSF concentrations in Alzheimer's disease, normal pressure hydrocephalus and healthy controls. J Psychiatr Res, 2007. 41(5): p. 387-94; Zhang, J., et al., CSF multianalyte profile distinguishes Alzheimer and Parkinson diseases. Am J Clin Pathol, 2008. 129(4): p. 526-9; Li, G., et al., Cerebrospinal fluid concentration of brain-derived neurotrophic factor and cognitive function in non-demented subjects. PLoS One, 2009. 4(5): p. e5424). In addition, a post-mortem study showed that AD patients have decreased BDNF mRNA in the hippocampus compared to healthy controls (Phillips, H. S., et al., BDNF mRNA is decreased in the hippocampus of individuals with Alzheimer's disease. Neuron, 1991. 7(5): p. 695-702). Increasing BDNF with DBS may contribute to improving memory and neural plasticity. Indeed, the direct administration of entorhinal BDNF in rodents and non-human primates reverses neuronal atrophy and ameliorates age-related cognitive impairment (Nagahara, A. H., et al., Neuroprotective effects of brain-derived neurotrophic factor in rodent and primate models of Alzheimer's disease. Nat Med, 2009. 15(3): p. 331-7).

In parallel to the increase in BDNF, hippocampal VEGF expression was increased at 2.5 h. VEGF is a well-known cellular mitogen and a vascular growth factor. In addition to its pro-angiogenic activity, studies have revealed neurotrophic and neuroprotective potentials of this growth factor (Tillo, M., C. Ruhrberg, and F. Mackenzie, Emerging roles for semaphorins and VEGFs in synaptogenesis and synaptic plasticity. Cell Adh Migr, 2012. 6(6): p. 541-6). VEGF is implicated in the differentiation and formation of blood vessels in the brain as well as in neurogenesis (Maurer, M. H., et al., Expression of vascular endothelial growth factor and its receptors in rat neural stem cells. Neurosci Lett, 2003. 344(3): p. 165-8; During, M. J. and L. Cao, VEGF, a mediator of the effect of experience on hippocampal neurogenesis. Curr Alzheimer Res, 2006. 3(1): p. 29-33). Abnormal regulation of VEGF expression has been reported in the pathogenesis of AD (Ruiz de Almodovar, C., et al., Role and therapeutic potential of VEGF in the nervous system. Physiol Rev, 2009. 89(2): p. 607-48). Despite the elevation of hippocampal BDNF and VEGF expression, we did not observed any change in GDNF expression following fornix DBS.

As fornix DBS is currently being investigated for its potential in treating AD, we were also interested in studying the effects of DBS on proteins involved in the formation of Aβ plaques and neurofibrillary tangles (Selkoe, D. J., Toward a comprehensive theory for Alzheimer's disease. Hypothesis: Alzheimer's disease is caused by the cerebral accumulation and cytotoxicity of amyloid beta-protein. Ann N Y Acad Sci, 2000. 924: p. 17-25); we did not observe significant changes in the expression of APP, tau and ptau proteins (FIG. 5) with 1 hour of stimulation. Moreover, we were unable to detect a signal for Aβ, likely due to low levels of protein expression. Indeed, previous studies have shown that young rats, similar to those used in this study, have very low concentrations of both Aβ-40 and Aβ-42, that are undetectable by Western Blot (Silverberg, Miller, et al. 2010, Silverberg, Messier, et al. 2010).

### Conclusion

We have shown that one hour of fornix DBS activated the hippocampus and led to an increase in neurotrophic factors as well as markers of synaptic plasticity, which are all known to play crucial roles in memory functions. Changes in the expression of these proteins could contribute to improvement of memory after fornix stimulation.

While the examples have described the use of an electrode to stimulate the fornix region of the brain, it is to be appreciated that the invention is not strictly limited to the stimulation of this brain region. Rather, the present invention could be used to stimulate any neural tissue in which it is desired to regulate protein concentrations, including cortical and subcortical areas of the brain, the spinal cord, and peripheral nerves. It is also to be appreciated that more than one electrode could be implanted, so that more than one brain region could be stimulated during treatment.

It is to be appreciated that the invention is not limited to the exemplary electrode contructions that have been described and illustrated. Rather, any implantable device capable of delivering an electric current to the subject's neural tissue could be used. For example, the implantable neurostimulator device described in U.S. Pat. No. 8,380,304 to Lozano could be used with the present invention. U.S. Pat. No. 8,380,304 is hereby incorporated by reference in its entirety.

Although the detailed description has focused on the use of deep brain electrical stimulation to regulate protein levels in the brain, the invention is not limited solely to the regulation of proteins. For example, the invention also includes within its scope the use of electrical stimulation to reduce the concentration of non-protein toxic molecules in the neural tissue of a subject. In this regard, a skilled artisan will appreciate that mechanisms described above in relation to the clearance of proteins, such as opening of the blood brain barrier and activation of the inflammatory response, may also assist in the clearance of non-protein toxic molecules from the brain.

It will be understood that, although various features of the invention have been described with respect to one or another of the embodiments of the invention, the various features and embodiments of the invention may be combined or used in conjunction with other features and embodiments of the invention as described and illustrated herein.

Although this disclosure has described and illustrated certain preferred embodiments of the invention, it is to be understood that the invention is not restricted to these particular embodiments. Rather, the invention includes all embodiments which are functional, electrical or mechanical equivalents of the specific embodiments and features that have been described and illustrated herein.

1. A method of reducing the concentration of one or more toxic molecules in neural tissue of a human or animal subject, the method comprising:
selecting a neural region where the concentration of the one of more toxic molecules is to be reduced;
implanting an electrode into the neural tissue of the subject in or adjacent to the selected neural region;
configuring the electrode to deliver an electric current selected to reduce the concentration of the one of more toxic molecules; and
delivering the electric current to the neural tissue in the selected neural region through the electrode.

2. The method according to claim 1, wherein the method is used to treat or prevent a neurodegenerative disorder selected from the group consisting of Alzheimer's disease, Parkinson's disease, amyotrophic lateral sclerosis, Huntington's disease, post injury neurodegeneration, post stroke neurodegeneration, and prion disease.

3. The method according to claim 1, wherein the one or more toxic molecules comprise hyperphosphorylated tau, amyloid beta, synuclein, trinucleotide repeat related proteins including mutant huntingtin protein, or misfolded prion protein.

4. The method according to claim 1, wherein the electric current is selected to enhance clearance of the one or more toxic molecules and/or to reduce production of the one or more toxic molecules.

5. The method according to claim 1, wherein the electric current is selected to enhance transport of the one or more toxic molecules out of the neural tissue.

6. The method according to claim 1, wherein the electric current is selected to enhance inflammation.

7. The method according to claim 1, wherein the electric current is selected to open the blood brain barrier of the subject, to enhance clearance of the one or more toxic molecules.

8. The method according to claim 1, further comprising:
monitoring the concentration of the one or more toxic molecules on an ongoing basis using one or more sensors; and
adjusting the electric current in response to feedback from the one or more sensors.

9. The method according to claim 1, wherein the electric current is selected to activate microglia and astrocytes; to enhance expression of trophic and synaptic molecules; and to promote clearance of the one or more toxic molecules.

10. A method of regulating the clearance of one or more proteins in neural tissue of a human or animal subject, the method comprising:
selecting a neural region where the clearance of the one of more proteins is to be regulated;
implanting an electrode into the neural tissue of the subject in or adjacent to the selected neural region;
configuring the electrode to deliver an electric current selected to regulate the clearance of the one of more proteins; and
delivering the electric current to the neural tissue in the selected neural region through the electrode.

11. The method according to claim 10, wherein the electric current is selected to reduce or enhance the stability of the one or more proteins.

12. The method according to claim 10, wherein the electric current is selected to reduce or enhance proteolysis of the one or more proteins.

13. The method according to claim 10, wherein the electric current is delivered continuously or intermittently for a period of at least 1 hour.

14. The method according to claim 13, wherein the period is at least 1 year.

15. The method according to claim 10, wherein the electrode is permanently implanted into the neural tissue of the subject for chronic treatment of a neurodegenerative disorder.

16. The method according to claim 10, further comprising:
determining a concentration of the one or more proteins in the neural tissue; and
adjusting the delivery of the electric current based on the determined concentration.

17. The method according to claim 16, wherein the concentration of the one or more proteins is determined by testing a plasma sample, testing a cerebrospinal fluid sample, or preparing a brain image.

18. A method of enhancing the expression of one or more proteins in neural tissue of a human or animal subject, the method comprising:
selecting a neural region where the expression of the one of more proteins is to be enhanced;
implanting an electrode into the neural tissue of the subject in or adjacent to the selected neural region;
configuring the electrode to deliver an electric current selected to enhance the expression of the one of more proteins; and
delivering the electric current to the neural tissue in the selected neural region through the electrode;
wherein the one or more proteins comprise Growth Associated Protein 43, synaptophysin, and/or α-synuclein.

19. The method according to claim 18, wherein the selected neural region is the fornix.

20. The method according to claim 19, wherein the electric current is selected to activate the hippocampus and/or to stimulate growth of the hippocampus.

21. The method according to claim 19, wherein the electric current is delivered in pulses at a voltage of 2.5 V, a pulse width of 90 msec, and a frequency of 130 Hz for at least 1 hour.

22. The method according to claim 19, wherein the method is used to improve hippocampus dependent memory in an Alzheimer's patient.
